(19) United States
(12) Patent Application Publication
(10) Pub. No.: US 2011/0092706 A1
(43) Pub. Date: Apr. 21, 2011

(54) Process for the manufacture of enantiomerically pure aryloctanoic acids as aliskiren

(75) Inventor: Milan Soukup, Sarasota, FL (US)

(73) Assignee: CarboDesign LLC, Sarasota, FL (US)

(21) Appl. No.: 12/925,154

(22) Filed: Oct. 15, 2010

Related U.S. Application Data
(60) Provisional application No. 61/279,360, filed on Oct. 21, 2009.

(51) Int. Cl. C07C 231/12; C07D 491/052; C07D 211/42

(52) U.S. Cl. 546/116; 546/220; 546/219; 564/157

(57) ABSTRACT

The present invention relates to a novel manufacturing process and novel intermediates useful in the synthesis of pharmaceutically active compounds, especially rennin inhibitors such as Aliskiren. The invention describes a preparation of enantiomerically pure 8-aryloctanoic acids of general formula I from readily available key intermediate, a novel bicyclic compound of formula IV.

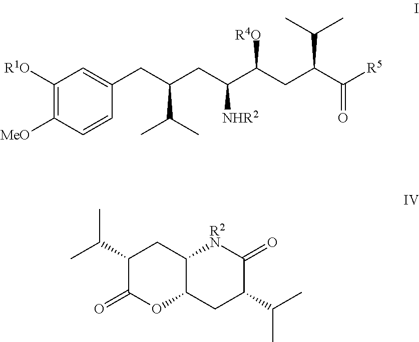

This application claims priority to U.S. Provisional Application Ser. No. 61/279,360, filled Oct. 21, 2009.

### BACKGROUND OF THE INVENTION

8-Aryloctanoic acids of a general formula I, having the 2S,4S,5S,7S-configuration

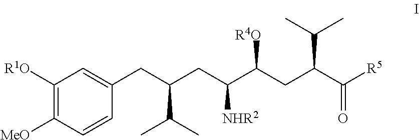

especially compound such as Aliskiren, wherein R¹ represents CH₃OCH₂CH₂CH₂—, R² and R⁴ hydrogen and R⁵—NHCH₂C(CH₃)₂CONH₂, (INN name: 5-amino-N-(2-carbamoyl-2-methylpropyl)-4-hydroxy-2-isopropyl-7-[4-methoxy-3-(3-methoxypropoxy)benzyl]-8-methyl-nanoamide), are excellent new antihypertensive which interfere with the rennin-angiotensin system.

After discovery of the biological activity of these compounds of general formula I in 1994, the first synthesis of Aliskiren has been also disclosed (U.S. Pat. No. 5,559,111 and EP 0 678 503). Since Aliskiren contains 4 chiral centers, synthesis of enantiomerically pure compound is very complex. After 2001 many patents and publications have been filed or published claiming alternative routes to Aliskiren (WO 01/09083, WO 01/09079, EP 1 215 201, WO 02/02508, WO 02/02500, WO 02/02487, WO 02/08172, WO 02/092828, WO 02/02500, WO 03/103653, UK 2 431 640, GB 2 431 641, GB 2 431 642, GB 2 431 643, GB 2 431 644, GB 2 431 645, GB 2 431 646, GB 2 431 647, GB 2 431 48, GB 2 431 649, GB 2 431 650, GB 2 431 651, GB 2 431 652, GB 2 431 653, GB 2 431 654, WO 2005/054177, WO 2005/090305, WO 2005/051895, WO 2006/131304, WO2006/095020, WO2006/024501, WO2007/054254, WO2007/039183, EP 2 062 874, EP 1958 666, WO 2007/006532, WO2007/045420, WO2008/155338, WO2008/119804, CA 2 634 513, WO2007/048620, WO2007/118681, US2009/0076062, WO2010/010165, EP2189442, WO2009/049837, Tetrahedron Letters 2000, 41, 10085, ibid. 2000, 41, 10091, ibid. 2001, 42, 4819, Drugs Fut. 2001, 1139, J. Org. Chem. 2002, 67, 4261, Helv. Chim Acta 2003, 86, 2848, Tetrahedron Letters 2005, 46, 6337, J. Org. Chem. 2006, 71, 4766, Organic Process & Develop 2007, 11, 584, Tetrahedron Letters 2008, 49, 5980 and Org. Lett. 2010, 12, 1816) Nevertheless, none of them fulfill requirements for a short and cost effective manufacturing process.

### SUMMARY OF THE INVENTION

The present invention discloses a novel, cost effective process for the manufacture of enantiomerically pure compounds of general formula I, specifically of Aliskiren as shown in Scheme 1:

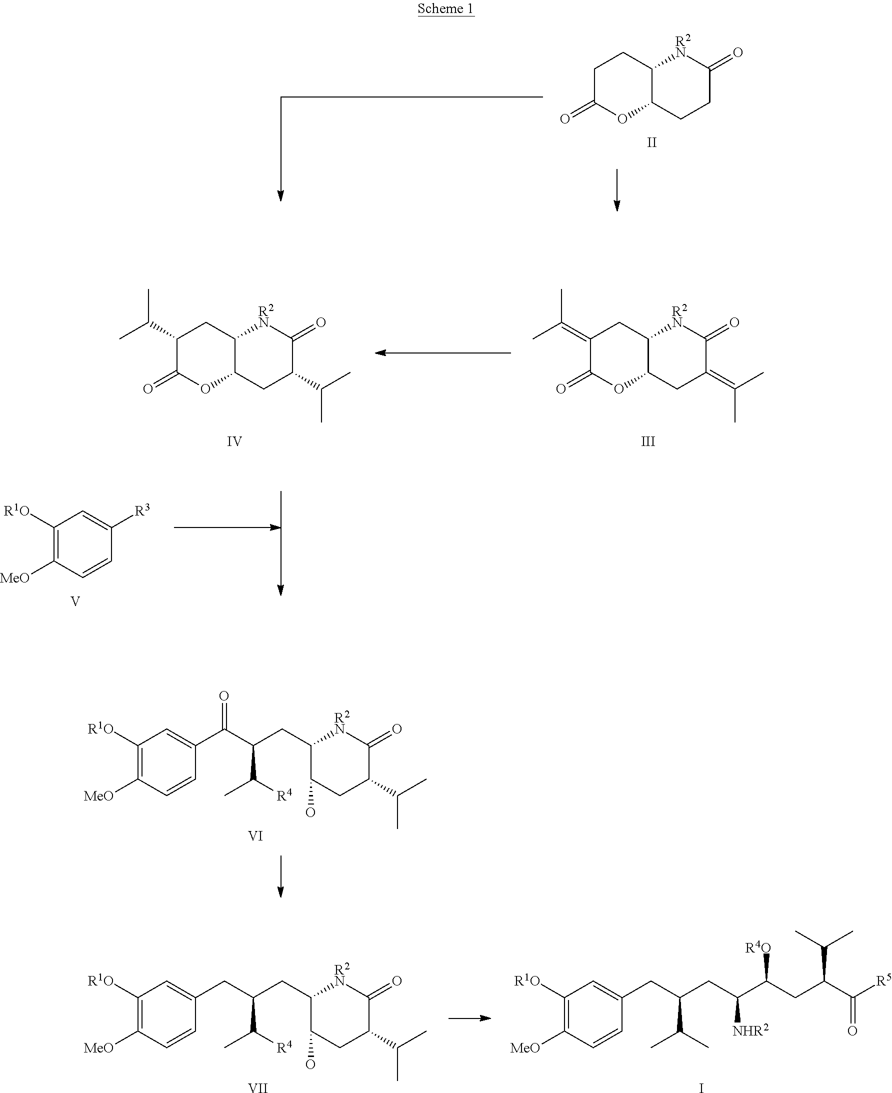

It has been unexpectedly found that compounds of general formula I and the corresponding intermediates thereof (formulas IV, VI and VII) can be efficiently prepared in only a few steps from a novel chiral bicyclic compound of formula II which is accessible from various inexpensive materials as shown in Scheme 2:

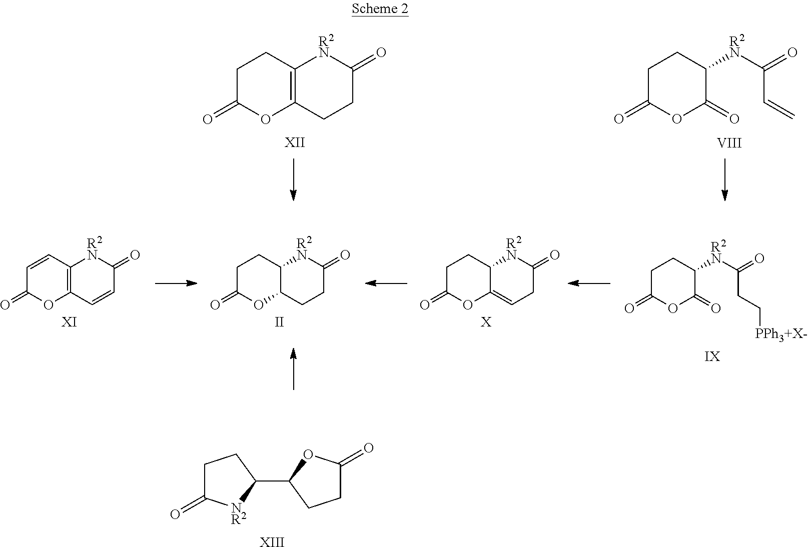

According to this invention the enantiomerically pure compound of formula I containing 4 chiral centers can be prepared from bicyclic compound of formulas IV (Scheme 1). The bicyclic compound IV is accessible from a new compound of formula II which contains only two chiral centers: With these two chiral centers in an alkylation step the third and fourth chiral centers can be formed diastereoselective leading to compound of formula IV. In all previously patented concepts. the potential of these two new compounds of formulas II and IV has never been considered

The invented process, as shown in Schemes 1 and 2 for enantiomerically pure compounds with the specific configuration, can also be applied for preparation of racemic compounds of formulas II, III, IV, VI and VII which can be alternatively subjected at any stage of the synthesis to a resolution step.

### DETAILED DESCRIPTION OF THE INVENTION

The present invention claims a new process for the preparation of a compound of general formula I

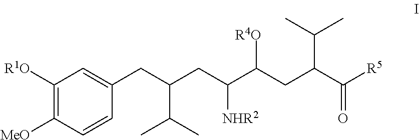

wherein
R¹ represents hydrogen, linear or brunched C₁₋₆ alkyl, C₁₋₆ alkoxy-C₁₋₆ alkyl, aryl, alkylaryl, arylalkyl, preferably CH₃OCH₂CH₂CH₂—, or acyl or carbamoyl, trifluoroacetyl, Mesyl, Tosyl, trifluoromethanesulfonyl, trialkylsilyl or alkylarylsilyl;
R² represents hydrogen, arylalkyl, alkoxy, aryloxy, arylalkoxy, alkylaryloxy, trialkylsilyl, alkylarylsilyl, with heteroatom(s) substituted arylalkyl, preferably benzyl, mono-, di- or tri-methoxybenzyl, or other amino protective group, in particular one which together with N forms an amide or carbamate as —C(O)-alkyl, —C(O)-aryl, —C(O)-alkylaryl, —C(O)-arylalkyl, —C(O)—Oalkyl, —C(O)—Oaryl, —(O)C-Oalkylaryl, —C(O)—Oarylalkyl, preferably —C(O)Obenzyl (Cbz) or —C(O)O-tert.butyl (BOC), acetyl, trifluoroacetyl and formyl;
R⁴ represents hydrogen, arylalkyl, preferably benzyl, substituted benzyl, acyl or carbamoyl, as e.g. —C(O)-alkyl, —C(O)-aryl, —C(O)-alkylaryl, —C(O)-arylalkyl, —C(O)-Oalkyl, —C(O)—Oaryl, —(O)C-Oalkylaryl, —C(O)—Oarylalkyl, preferably —C(O)Obenzyl (Cbz) or —C(O)O-tert.butyl (BOC), formyl, trifluoracetyl, or trialkylsilyl or alkylarylsilyl;
R⁵ represents hydroxy, linear or brunched C₁₋₆ alkyloxy, aryloxy, alkylaryloxy, arylalkyloxy or trialkylsilyloxy or alkylarylsilyloxy, halogen, preferably Cl or Br, —NH₂, —NMe₂ and preferably —NHCH₂C(CH₃)₂CONH₂;
comprising following steps:
a) reaction of the compound of formula II, wherein R² is the same as defined above for compound of formula I,

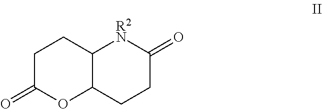

with a strong organic or inorganic base, preferably organic lithium or sodium amides as e.g. LDA or LiHMDS or NaHMDS, and then with either
aa) acetone followed by dehydration and isomerisation step providing compound of formula III,

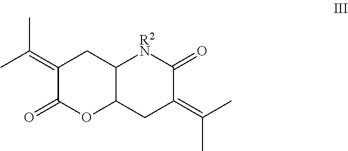

wherein R² is the same as defined above for compound of formula I, which is then hydrogenated or reduced converting both double bonds into single bonds or
bb) isopropyl halide, preferably isopropyl bromide or iodide,
providing a compound of formula IV

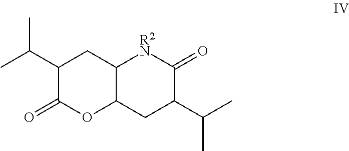

wherein R² is the same as defined above for compound of formula I;
b) reaction of the compound of formula IV with compound of formula V

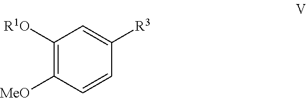

wherein R¹ is the same as defined for compound of formula I and
R³ is a metal containing group such as —Li, —Na, Znhalide, magnesate or -Mghalide, boronic acid as —B(OH)₂, -Cehalide, -Cuhalide or dicuprate, preferably Li or MgBr, providing a compound of formula VI

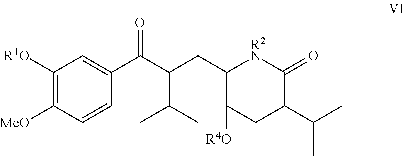

wherein R¹, R² and R⁴ are the same as defined for compound of formula I;
c) reduction or/and hydrogenation of C(8)-oxo group in the compound of formula VI, simultaneously or in two separate steps, to a compound of formula VII

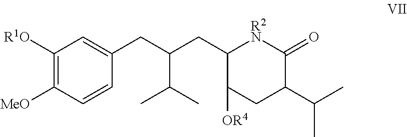

wherein R¹, R² and R⁴ are the same as defined for compound of formula I;
d) hydrolysis of the lactam of formula VII and eventual protection of C(5)-amino group followed by either
   protection of the C(4)-hydroxyl group and activation of the free carboxylic acid function with an appropriate peptide coupling reagent or
   formation of a 5-membered lactone ring with C(4)-hydroxyl group and reaction with R⁵—H, wherein R⁵ is the same as defined for compound of formula I, preferably with NH₂CH₂C(CH₃)₂CONH₂.

Depending on the choice of starting materials the compounds can be present in the form of one possible isomers or a mixture thereof, for example as enantiomerically pure compound or as isomer mixtures, such a racemates, diastereomer mixtures etc., depending on the number of asymmetric carbon atoms.

As further embodiment of the invention the racemic compounds of formulas II, IV, VI and VII can be subjected at any stage of the synthesis to a resolution or separation step using (chiral) agent or subjected, if possible, to an enzymatic step or to another separation method known as e.g. preparative HPLC or SMB etc. As the resolution agent any chiral acid or base, as commonly used for resolution of nitrogen- or alcohol- or carboxylate-containing compounds (e.g. TCI Reagent Guide 2009-2010, p. 50-56), can be used.

In a preferred embodiment of the invention preparation of the enantiomerically pure compound of general formula I can be carried out as shown in Scheme 1: The enantiomerically pure bicyclic compound of formula II,

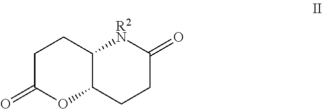

possessing two chiral centers, can be deprotonated with a strong organic or inorganic base, preferably LDA or LiHMDS or NaHMDS, in inert organic solvent, preferably THF at temperature between −50-0° C. and the enolate formed then stereoselectively alkylated in situ with isopropyl halide, preferably isopropyl iodide, leading to compound of formula IV. The alkylation with isopropyl halide under basic condition proceeds with high stereo selectivity control at third and fourth chiral centers resulting in formation of one single stereo isomer IV with following configuration:

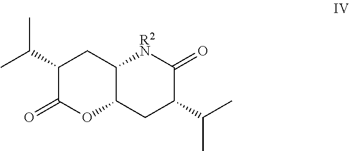

The chiral compound of formula IV can also be prepared in process comprising deprotonation of the chiral compound of formula II with either a weak organic or inorganic base or a strong organic or inorganic base, preferably LDA or LiHMDS or NaHMDS, in inert organic solvent, preferably THF at temperature between −50-0° C. and subsequent reaction of the enolate formed, itself or in the presence of activating Lewis acid catalysts as e.g. borontrifluoro etherate or BiCl₃ or CeCl₃, with acetone. After dehydration step, which can be accomplished in many different ways as well known in the literature, preferably MsCl/Et₃N, a compound of formula III, or double bond regio isomers thereof, are then subjected to either hydrogenation or chemical reduction, preferably heterogeneous hydrogenation in presence of 10% Pd—C or PtO₂ in the presence of a tert. amine base as Et₃N at rt or slightly elevated temperature and elevated pressure, converting both double bonds into single. The hydrogenation of the compound of formula III proceeds with high stereo selective control giving again compound of formula IV as a single diastereomer with the desired configuration as shown above.

The enantiomerically pure compound of formula IV can be reacted with compound of formula V in analogy as reported in U.S. Pat. No. 5,559,111 (p. 78) or WO2007/045420 (p. 64, 67 and 68) providing a chiral compound of formula VI

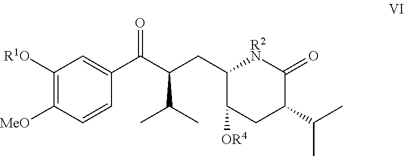

which is then subjected to reduction or hydrogenation step giving compound of formula VII.

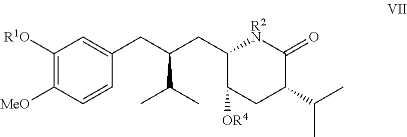

The compound of formula V, wherein R³ is metallic, especially an alkali or earth alkali metallic radical, as e.g. lithium, sodium, potassium or a group of the formula Mg-halogen, preferably bromine, is prepared from the corresponding aromatic halide (a compound of formula V, wherein R³ is a halide, preferably bromide) and is used in situ in an ethereal solvent, such as THF or toluene at a temperature range of −78° C. to 0° C. similar as reported in Novatis patent (p. 30, 78 and 82 U.S. Pat. No. 5,559,111). To reach good selectivity in Grignard reaction, Lewis acid catalysts, which are compatible with organometallic reagent, as e.g. boron trifluoro etherate, or preferably Ce-halides as CeCl₃ can be added, prior the reaction of compound of formula V with compound of formula IV.

The reduction of C(8)-oxo group in the compound of formula VI can be achieved simultaneously or in two separate steps analogues as reported in WO2007/045420 (p. 30-35): Typically, hydrogenation or/and reduction with a hydride can be employed and whenever the term “reduction” is used in general terms in this application. It might include both a hydrogenation and/or reduction with hydride (e.g. Synthesis 1987, 736 and J. March, j. Wiley&Sons, NY 1992, Advanced Org. Chemistry p. 1209-1211). A preferred reduction method is hydrogenation in the presence of homogeneous or heterogeneous hydrogenation catalysts. Catalyst for hydrogenation can be PtO₂ or 10% Pd—C or even Ra—Ni in polar or apolar solvents, preferably glacial acetic acid or alcohols, at rt or slightly elevated temperature under normal pressure or until 10 bar pressure. Also chemical reduction with alkali or earth alkali metal hydrides, preferably sodium or lithium borohydride, DIBAH, triethylaluminium can be carried out. Preferably this reduction can also be accomplished with trialkylsilane, preferably triethylsilane, in protic or aprotic solvents, preferably chlorinated hydrocarbons as dichloromethane, in the presence of acids, preferably trifluoromethane sulphonic acid, trifluoro acetic acid or even Lewis acids as e.g. bortrifluoro etherate, ZnCl₂, ALCl₃, TiCl₄, Yb(OTf)₃ (TCI Reagent Guide 2009, p. 16-17, J. Org. Chem. 1973, 38, 2675, ibid. 1978, 43, 374, Synthesis 1986, 770), at reaction temperature between −78 C until reflux, preferably rt.

The compound of formula VII can be converted into the compound of formula I according to steps comprising opening of the 6-membered lactam ring, protection of C(5)-amino group and reaction of the free carboxylic acid or ester thereof with R⁵—H as commonly known for preparation of amides using known coupling methods (as already described in U.S. Pat. No. 5,559,111 and TCI Reagent Guide 2009, p. 85-89). The C(5)-amino protected free carboxylic acid (step d)) can be reacted with R⁵—H, preferably 3-amino-2,2-dimethylpropionamide, according to standard peptide coupling method as also described for this step in U.S. Pat. No. 5,559,111 on page 22-25 or, as reported in Houben-Weyl, Methoden der organischen Chemie, 4^{th} Edition, Synthese von Peptiden1, Volume 15/II (1974), Volume IX (1955), Volume E 11 (1985), Gerge Thieme Verlag, Stuttgart, The Peptides, (e. Gross and J. Meienhofer) Volume 1 and 2, Academic Press, London 1979/1980 or M. Bodansky Principles of Peptide Synthesis, Springer Verlag, Berlin 1984. The condensation of the free carboxylic acid with amine can be carried out in the presence of one of the coupling agents as e.g. DCC or other dialkyl carbodiimides, carbonyldiimidazole, 1,2-oxazolinium compounds, e.g. 2-ethyl-5-phenyl-1,2-oxazolium-3″-sulphonate and 2-tert.-butyl-5-methylisoxazolium perchlorate, or a suitable acylamino compound, e.g. 2-ethoxy-1-ethoxy-carbonyl-1,2-dihydroquinoline, or activated phosphoric acid derivatives, bis(2-oxo-3-oxazolidinyl)phosphinic acid chloride or 1-benzotriazolyloxy-tris(dimethylamino)phosphonium hexafluorophosphate etc.

In alternative approach the compound of formula VII can be subjected opening of the 6-membered lactam ring, protection of C(5)-amino group, followed by a spontaneous lactonization leading to already known 5-membered lactone as reported in GB 2 431 649, GB 2 431 651 or WO2006/024501. As was shown in U.S. Pat. No. 5,559,111 the lactone can be efficiently converted into compound of formula I.

In the preferred embodiment of the invention, C(5)-amino protected 5-membered lactone (step d)) is reacted with NH₂CH₂C(CH₃)₂CONH₂ as reported in EP-A-678 503 (p. 124, 130 and 131) or WO02/02508 (example H1 p. 35, preparation of J1) or U.S. Pat. No. 5,559,111 (example 83, page 22-25) or WO2006/024501 (page 46-47).

As a further embodiment of the invention, instead of the above described reaction of organometallic compound of formula V with compound of formula IV, polarity of both reaction components can be reversed: According to this approach 6-membered ring lactone of formula IV has been first selectively opened and the free carboxylic acid activated by converting into corresponding acid chloride or bromide or mixed anhydride, preferably chloride or —OCOCF₃ or —OMesyl or —OSO2CF₃, which can undergo Friedel-Crafts reaction with a compound of formula V, wherein R¹ is —COCF₃, trifluoromethane sulphonyl-, Tosyl- or Mesyl- (Tetrahedron Letters 2002, 43, 7077) and R³ is hydrogen, in the presence of Lewis acid, as commonly used for F—C.-reactions e.g. aluminium chloride, bortrifluoro etherate, metal halide (preferably Al-, Zn-, lanthanide- or Bi-halides or aluminium dodecatungsttophosphate etc., s. Tetrahedron Letters 2003, 44, 2937, ibid. 2003, 44, 5343, Tetrahedron 2004, 60, 10843). After F.-C. reaction the reaction sequence, as shown in Scheme 1, can be accomplished in the same way and, in any stage of the synthesis, the protective/deactivation R¹-group, as e.g. trifluoromethane sulphonyl, Mesyl or Tosyl, can be removed and replaced by a group as defined for compound of formula I. As solvent for Friedel-Crafts-reaction common aprotic inert organic solvent, preferably chlorinated hydrocarbons as methylenechloride or aliphatic hydrocarbons as hexane or heptane, can be used.

As a further embodiment of the invention the starting compound of formula II can be prepared in many different ways, preferably as shown in Scheme 2: Starting with inexpensive chiral (S)-glutamic acid, treatment with acylating reagent such as e.g. acryloyl chloride or anhydride, provides cyclic anhydride of formula VIII (J. Org. Chem. 1987, 52, 5331 or Archiv of Pharm. Res. 2004, 27, 151 and Org. Process Res. & Develop. 2004, 8, 72) which can be converted into novel bicyclic compound of formula X using different reaction conditions: As shown in Scheme 2, the compound of formula VIII can be treated with PPh₃ or PPh₃ hydrobromide yielding Wittig salt of formula IX which undergoes under basic conditions cyclization giving a compound of formula X. For the cyclization step also another reagents can be used as e.g. Horner- or Peterson-reagent (s. TCI Reagent Guide 2009, p. 90-93). Instead of acryloyl chloride another agents containing C₃-fragment with an incorporated reagent necessary for the cyclization step, can also be used. Reduction, preferably hydrogenation, of the double bond in compound of formula X leads stereoselectively to the compound of formula II.

The chiral compound of formula II can also be obtained from known compounds of formula XI (Microbiological Rev. 1966, 60(3), 483 and J. Heterocyclic. Chem. 1993, 30, 671) or XII via asymmetric (preferably homogeneous) hydrogenation.

As a further embodiment of the invention the chiral compound of formula II can be prepared from a known compound of formula XIII (WO2008/119804, p. 4), wherein R² is hydrogen or any N-protective group, preferably the same one as defined for compound of formula II, via reactions sequence comprising opening of lactam and lactone rings e.g. under basic condition, preferably with alkali metal hydroxide, followed by protection of the amino group and selective re-closure, preferably under acidic conditions. The initial sequence can also be reversed in the way that nitrogen in the compound of formula XIII can be first protected with common protective group as e.g. BOC or Cbz-groups facilitating subsequent base catalyzed lactam opening, accompanied with simultaneous opening of the lactone ring followed by controlled double ring closure, preferably under acidic conditions, leading to the compound of formula II.

In this invention a characteristic of protective groups is that they can be removed readily (without the occurrence of undesired secondary reactions) for example by solvolysis, reduction, or alternatively under physiological conditions (as e.g. enzymatic cleavage or formation). Different protective groups can be selected so that they can be removed selectively at different stages of the synthesis while other protective groups remain intact. The corresponding alternatives can be selected readily by a person skilled in the art from those given in the standard reference works mentioned in literature (as e.g. Mc Omie “Protective Groups in Organic Chemistry” or Green et al. “Protective Groups in Organic Synthesis”) or in the description or in the claims or the Examples.

When referring to compounds described in the present invention, it is understood that references are also being made to salts thereof.

The example are provided to illustrate particular aspects of the disclosure and do not limit the scope of the present invention as defined by the claims.

### EXAMPLES

Determination of optical purity was carried out with HPLC using chiral columns as Chiralcel OJ-H, Chiralpak AS-H or Chiralpak AD-H from Daicel Chem. Ind. In some cases the optical purity was also determined with NMR-Spectroscopy using chiral Eu-shift reagent. If not mentioned otherwise, all evaporation are performed under reduced pressure, preferably between 5-50 Torr. The structure of final products, intermediates and starting materials is confirmed by standard analytical methods, e.g. spectroscopic characteristics as MS or NMR or IR. Abbreviation used are those conventional in the art.

### Example 1

### Preparation of Compound (IVa) from Compound (IIa)

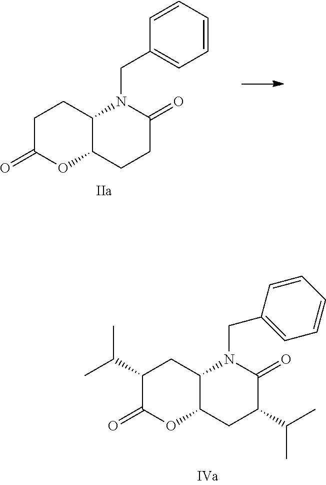

A 1.56 M solution of butyllithium in hexane (150 ml) was added over ca. 15 min under good stirring in an atmosphere of dry nitrogen to a cooled (−40° C.) solution of dry diisopropylamin (25 g) in dry THF (200 ml). To this solution, stirred for ca. 10 min at −40° C., a solution of compound (IIa) as shown above (26 g), dissolved in dry THF (75 ml), was then added at a rate that the reaction temperature did not exceed −40° C. Stirring was continued at −40° C. for ca. 30 min. To the slurry diisopropyl iodide (50 g) was added slowly and the temperature kept at under −40° C., the reaction mixture stirred then for another 3 hrs, wormed up to ca.-10° C. and stirred for another 2 hrs. The reaction mixture was poured on water (500 ml), the aqueous phase extracted 4 times with ethylacetate (4×150 ml), the combined organic phases washed with brine, dried with sodium sulfate, filtered and evaporated under reduced pressure providing the title compound (IVa) as a single diastereomer: 25 g (73% yield) as a colorless oil. MS (M⁺343), Anal. calculated for C₂₁H₂₉NO₃: C, 73.44; H, 8.51; N, 4.08; O 13.97. Found: C, 73.01; H, 8.22; N, 4.04; O 14.20.

### Preparation of Compound (Ia, Aliskiren) from Compound (IVa)

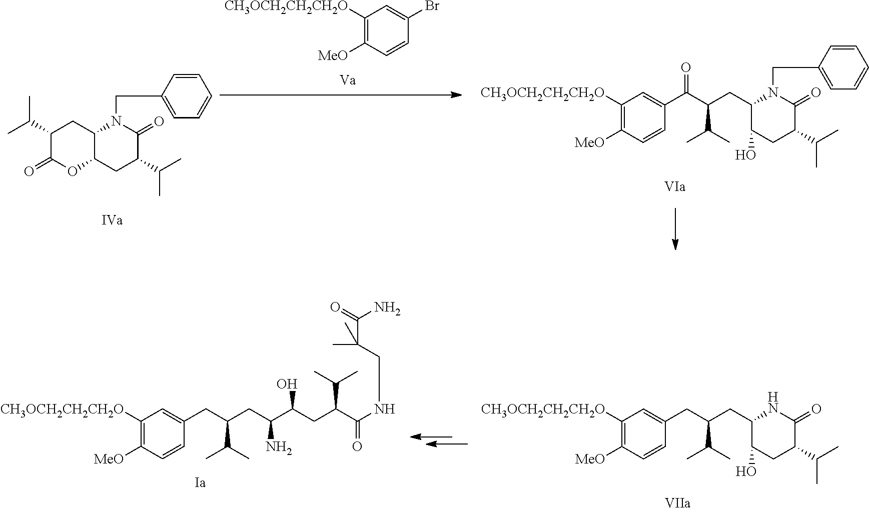

### Example 2

### a1) Preparation of Compound (VIa) From (IVa) Via Grignard Reagent

Several crystals of iodine were added to a suspension of magnesium turnings (5 g) in THF (150 ml) and the mixture was stirred at rt under nitrogen for ca. 3 hrs, then 10 drops of 1,2-dibromo butane were added and the mixture stirred for another 30 min. To this slurry compound (Va) (28 g), dissolved in dry THF (100 ml), was slowly added under stirring that the solvent started to reflux. When the addition was complete the reaction mixture was maintained under reflux for another hr. The reaction mixture was then cooled to it and added dropwise within a period of ca. 1 hr to a solution of compound (IVa) (28 g) and dry CeCl₃ (10 g), both dissolved in dry THF (150 ml) and cooled to −78° C. After addition the slurry was then stirred at −78° C. for another 2 hrs, acetic acid (25 ml) added at the same temperature and the mixture finally poured on saturated ammonium chloride solution (100 ml). After dilution with water (500 ml) the aqueous phase was extracted 4 times with ethyl acetate (4×100 ml), the combined organic phase washed once with saturated sodium bicarbonate solution (200 ml), dried over magnesium sulphate, filtrated and the solvents evaporated under reduced pressure providing the title compound (VIa) as a single diastereomer: crude 40 g (89% isolated yield) as a yellow oil. MS (M⁺539): Small sample of the crude (VIa) was purified by a column chromatography on silicagel, eluens: hexane/ethyl acetate (10:1.5): Anal. calculated for C₃₂H₄₅NO₆: C, 71.21; H, 8.40; N, 2.60; O 17.79. Found: C, 70.99; H, 8.61; N, 2.6; O 17.56.

### Example 3

### a2) Preparation of Compound (VIa) From (IVa) Using Lithium Reagent

To a solution of compound (Va) (30 g) dissolved in dry THF (250 ml), cooled to −78° C., 1.56 M solution of butyl lithium (80 ml) was slowly added under stirring that the reaction temperature was kept at −70° C., then stirred at this temperature for 1 hr before dry CeCl₃ (2 g), dissolved in dry THF (50 ml), was added. This reaction mixture was then added dropwise within a period of ca. 1 hr to a stirred solution of compound (IVa) (26 g), dissolved in THF (100 ml) and pre-cooled to −40° C. After stirring for 2 hrs at −40° C. the mixture was poured on saturated sodium bicarbonate solution (100 ml), the aqueous phase extracted 4 times with ethyl acetate (4×100 ml), the combined organic phases washed once with saturated sodium bicarbonate solution (200 ml), dried over magnesium sulphate, filtrated and the solvents evaporated under reduced pressure providing the title compound (VIa) as a single diastereomer: crude 25 g as a yellow oil having the same spectroscopic data as shown above in Example 2, part a1).

### Example 4

### b) Preparation of Compound (VIIa) from (VIa)

Compound (VIa) (5.5 g) was dissolved in glacial acetic acid (50 ml) and a few drops of concentrated HCl and 10% PtO₂ (400 mg) were added. The slurry was under intensive stirring hydrogenated under normal pressure at rt for ca. 3 hrs until more than 3 equivalents of hydrogen have been consumed. After filtration of the catalyst the solvent was evaporated under reduced pressure providing crude compound (VIIa) as a brawn oil: 5.5 g. This crude material was directly used for the next step (Example 5)

Instead of PtO₂ in acetic acid also other hydrogenation catalysts can be used as e.g. 10% Pd—C or Ra—Ni in ethanol or THF or ethyl acetate.

### Example 5

### c1) Preparation of Compound (Ia, Aliskiren) from Compound (VIIa)

Crude compound (VIIa) from the above experiment (Example 4) (5.5 g) was dissolved in aqueous ethanol (20 ml) and after addition of 2N-sodium (or alternatively lithium) hydroxide solution (10 ml) the solution was stirred for ca. 3 hrs at 50° C. and then ethanol evaporated under reduced pressure. The aqueous solution was diluted under stirring with a mixture of water/THF (3:1) to volume of ca. 50 ml and at rt chloro formate benzyl ester (1-4 ml) was slowly added to achieve complete Cbz-protection of the amino function (ca. 2 hrs). After acidification of the reaction mixture with conc. HCl to pH ca. 1-2 the Cbz-protected lactone was formed which was isolated by pouring the reaction mixture on water (200 ml) and extraction 3 times with ethyl acetate (3×50 ml), drying the organic phase with magnesium sulphate, filtration of the organic solution and evaporation under reduced pressure: The crude Cbz-protected lactone [(1S,3S)-1-((2S,4S)-4-isopropyl-5-oxo-tetrahydrofuran-2-yl)-3-[4-methoxy-3-(3-methoxypropoxy)-benzyl]-4-methyl-pentyl]-carbamic acid benzyl ester: (5.4 g) was directly converted into final compound (I).

A solution of the above prepared lactone (5.4 g), 3-amino-2,2-dimethyl propionamide (1.5 g) and 2-hydroxypyridine (1 g) in tert.-butyl methyl ether (10 ml), containing triethylamine (0.2 ml), was stirred for 18 hrs at 80° C., then cooled to rt and diluted with toluene (20 ml) and washed with 10% sodium hydrogen sulphate solution (ml). The organic phase was separated, washed once with water (50 ml), dried with magnesium sulphate, filtrated and evaporated under reduced pressure to give a yellow oil which was suspended in hexane (100 ml), slurry stirred a few min, filtrated and the filtrate evaporated under reduced pressure providing a foam of Cbz-protected derivative of Aliskiren (Ia): 4.5 g. The crude Cbz-protected Aliskiren (4.5 g) was the dissolved in a mixture of glacial acetic acid and ethanol (40 ml, 1:1) and after addition of 10% Pd—C under intensive stirring the slurry was hydrogenated under normal pressure at rt. The reaction mixture was then filtered to remove the catalyst, poured on water (100 ml) and pH adjusted with 37% sodium hydroxide solution to 10. The final product, Aliskiren was then extracted 4 times with dichloromethane (4×100 ml), the organic phase evaporated under reduced pressure providing crude Aliskiren (Ia): 4.0 g with identical analytical data as reported e.g. in EP 0 678 503 on p. 74, example 137: MS (M⁺552), Rf 0.33 on silicagel eluens: dichloromethane/methanol=8:2.

From the free base (Ia) the hemifumarate salt can be prepared, e.g. as described in U.S. Pat. No. 6,730,798 example J1.

### Example 6

### c2) Preparation of Compound (Ia, Aliskiren) from Compound (VIIa)

Crude compound (VIIa) from the above experiment (Example 4) (5.5 g) was dissolved in aqueous ethanol (20 ml) and after addition of 2N-sodium (or alternatively lithium) hydroxide solution (10 ml) the solution was stirred for ca. 3 hrs at 50° C., then glacial acetic acid (20 ml) was added and the solvents evaporated under reduced pressure to dryness. The residue was dissolved under stirring in mixture of water/THF (50 ml, 3:1) and the aqueous phase extracted 3 times with ethylacetate (3×100 ml), the organic solvent dried over magnesium sulphate, filtrated, solvent evaporated under reduced pressure and the residue dissolved in THF (20 ml). To this solution N,N-dimethyl aminopyride (0.3 g) triethylamine (3 g) and di-tert-butyldicarbonate (3 g) were added at rt and the mixture stirred for 24 hrs to achieve complete BOC-protection of the amino group. After acidification of the reaction mixture with glacial acetic acid (10 ml), the mixture was extracted with toluene/water mixture and the organic phase separated and under reduced pressure evaporated. The residue was taken in glacial acetic acid (20 ml), heated for 24 hrs at ca. 100° C., the acid removed under vacuum and purified by column chromatography (eluens: ethyl acetate/hexane 1:1): 4.1 g of desired BOC-protected lactone [(1S,3S)-1-(2S,4S)-4-isopropyl-5-oxo-tetrahydro-furan-2-yl)-3-[4-methoxy-3-(3-methoxypropoxy)-benzyl]-4-methyl-pentyl]-carbamic acid tert.-butyl ester. The analytical data were identical as reported e.g. in WO2006/024501, p. 58). The lactone was then converted into Aliskiren (Ia) as described above for Cbz-protected lactone in Example 5.

### Example 7

The Cbz-N-protected free acid from Example 5 or BOC-N-protected acid from Example 6 can also be directly reacted with 3-amino-2,2-dimethylpropionamide by standard peptide coupling method as reported in Houben-Weyl, Methoden der organischen Chemie, 4^{th} Edition, Synthese von Peptiden1, preferably e.g. DCC.

1. A compound of general formula II, 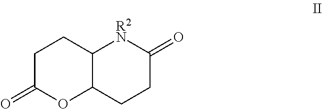
wherein R² represents hydrogen, alkyl, aryl, alkylaryl, arylalkyl, alkoxy, aryloxy, arylalkoxy, alkylaryloxy, trialkylsilyl, alkylarylsilyl, with heteroatom(s) substituted alkyl, aryl, alkylaryl, arylalkyl, preferably hydrogen, benzyl, mono-, di- or tri-methoxybenzyl, or
other N-protective group, in particular one which together with N forms an amide or carbamate, as —C(O)H, —C(O)-alkyl, —C(O)-aryl, —C(O)-alkylaryl, —C(O)-arylalkyl, —C(O)—Oalkyl, —C(O)—Oaryl, —C(O)—Oalkylaryl, —C(O)—Oarylalkyl, preferably formyl, acetyl, trifluoroacetyl, —C(O)Obenzyl (Cbz) or —C(O)O-tert.butyl (BOC);
and a salt thereof.

2. The compound according to claim 1, having the configuration as given in the formula

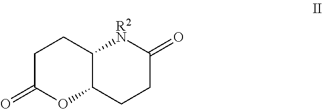

3. A compound of general formula III, 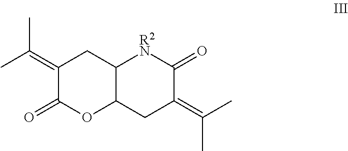
wherein R² is the same as defined for compound of formula II in claim 1, and a salt thereof.

4. The compound according to claim 3, having the configuration as given in the formula

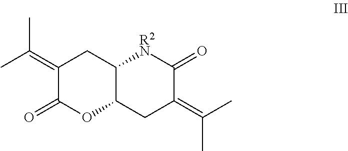

5. A compound of formula IV, 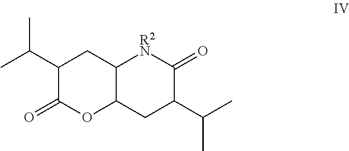
wherein R² is the same as defined for compound of formula II, wherein R² represents hydrogen, alkyl, aryl, alkylaryl, arylalkyl, alkoxy, aryloxy, arylalkoxy, alkylaryloxy, trialkylsilyl, alkylarylsilyl, with heteroatom(s) substituted alkyl, aryl, alkylaryl, arylalkyl, preferably hydrogen, benzyl, mono-, di- or tri-methoxybenzyl, or
other N-protective group, in particular one which together with N forms an amide or carbamate, as —C(O)H, —C(O)-alkyl, —C(O)-aryl, —C(O)-alkylaryl, —C(O)-arylalkyl, —C(O)—Oalkyl, —C(O)—Oaryl, —C(O)—Oalkylaryl, —C(O)—Oarylalkyl, preferably formyl, acetyl, trifluoroacetyl, —C(O)Obenzyl (Cbz) or —C(O)O-tert.butyl (BOC);
and a salt thereof.

6. The compound according to claim 5, having the configuration as given in the formula

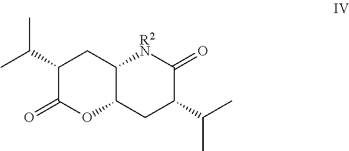

7. A compound of general formula VI, 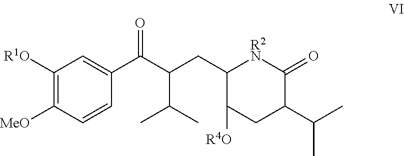
wherein
R¹ is hydrogen, linear or brunched C₁₋₆ alkyl, C₁₋₆ alkoxy-C₁₋₆ alkyl, aryl, alkylaryl, arylalkyl, preferably CH₃OCH₂CH₂CH₂— or acyl, carbamoyl, trifluoracetyl, mesyl, tosyl, trifluoromethanesulfonyl, trialkylsilyl or alkylarylsilyl;
R² is hydrogen, alkyl, aryl, alkylaryl, arylalkyl, alkoxy, aryloxy, arylalkoxy, alkylaryloxy, trialkylsilyl, alkylarylsilyl, with heteroatom(s) substituted alkyl, aryl, alkylaryl, arylalkyl, preferably hydrogen, benzyl, mono-, di- or tri-methoxybenzyl, or other N-protective group, in particular one which together with N forms an amide or carbamate, as —C(O)H, —C(O)-alkyl, —C(O)-aryl, —C(O)-alkylaryl, —C(O)-arylalkyl, —C(O)—Oalkyl, —C(O)—Oaryl, —C(O)—Oalkylaryl, —C(O)—Oarylalkyl, preferably formyl, acetyl, trifluoroacetyl, —C(O)Obenzyl (Cbz) or —C(O)O-tert.butyl (BOC);
R⁴ is hydrogen, arylalkyl, alkylaryl, preferably benzyl, mo-, di-, tri-methoxy benzyl, acyl, as —C(O)H, —C(O)-alkyl, —C(O)-aryl, —C(O)-alkylaryl, —C(O)-arylalkyl, —C(O)O—C₁₋₆alkyl, —C(O)O-alkylaryl, preferably formyl, acetyl, trifluoracetyl, —C(O)O-benzyl (Cbz), —C(O)O-tert.butyl (BOC), or trialkylsilyl or alkylarylsilyl;
and a salt thereof.

8. The compound according to claim 7, having the configuration as given in the formula

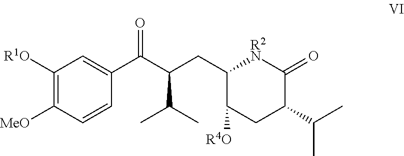

9. A compound of general formula VII, 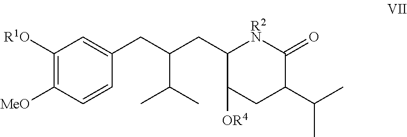
wherein R¹ is the same as defined for compound of formula VI in claim 7
R² represents hydrogen, alkyl, aryl, alkoxy, aryloxy, arylalkoxy, alkylaryloxy;
R⁴ is hydrogen;
and a salt thereof.

10. The compound according to claim 9, having the configuration as given in the formula

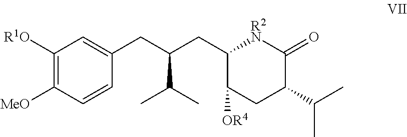

11. A process for the preparation of a compound of general formula I 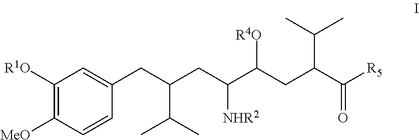
wherein
R¹ is hydrogen, linear or brunched C₁₋₆ alkyl, C₁₋₆ alkoxy-C₁₋₆ alkyl, aryl, alkylaryl, arylalkyl, preferably CH₃OCH₂CH₂CH₂— or acyl, carbamoyl, trifluoracetyl, mesyl, tosyl, trifluoromethanesulfonyl, trialkylsilyl or alkylarylsilyl;
R² is hydrogen, alkyl, aryl, alkylaryl, arylalkyl, alkoxy, aryloxy, arylalkoxy, alkylaryloxy, trialkylsilyl, alkylarylsilyl, with heteroatom(s) substituted alkyl, aryl, alkylaryl, arylalkyl, preferably hydrogen, benzyl, mono-, di- or tri-methoxybenzyl, or other N-protective group, in particular one which together with N forms an amide or carbamate, as —C(O)H, —C(O)-alkyl, —C(O)-aryl, —C(O)-alkylaryl, —C(O)-arylalkyl, —C(O)—Oalkyl, —C(O)—Oaryl, —C(O)—Oalkylaryl, —C(O)—Oarylalkyl, preferably formyl, acetyl, trifluoroacetyl, —C(O)Obenzyl (Cbz) or —C(O)O-tert.butyl (BOC);
R⁴ is hydrogen, arylalkyl, alkylaryl, preferably benzyl, mo-, di-, tri-methoxy benzyl, formyl, —C(O)-alkylaryl, —C(O)-aryl, —C(O)O—C₁₋₆alkyl, —C(O)O—-alkylaryl, —C(O)O-aryl, preferably —C(O)O-benzyl (Cbz), —C(O)O-tert.butyl (BOC), acetyl, trifluoroacetyl, or trialkylsilyl or alkylarylsilyl;
R⁵ is hydroxy, linear or brunched C₁₋₆ alkyloxy, aryloxy, C₁₋₆ alkylaryloxy, arylalkyloxy, trialkylsilyloxy or alkylarylsilyloxy, halogen, preferably chlorine or bromine or —NH₂, —NMe₂ or preferably —NHCH₂C(CH₃)₂CONH₂;
comprising following steps:
a) reaction of the compound of formula II, wherein R² represents hydrogen, alkyl, aryl, alkylaryl, arylalkyl, alkoxy, aryloxy, arylalkoxy, alkylaryloxy, trialkylsilyl, alkylarylsilyl, with heteroatom(s) substituted alkyl, aryl, alkylaryl, arylalkyl, preferably hydrogen, benzyl, mono-, di- or tri-methoxybenzyl, or
other N-protective group, in particular one which together with N forms an amide or carbamate, as —C(O)H, —C(O)-alkyl, —C(O)-aryl, —C(O)-alkylaryl, —C(O)-arylalkyl, —C(O)—Oalkyl, —C(O)—Oaryl, —C(O)—Oalkylaryl, —C(O)—Oarylalkyl, preferably formyl, acetyl, trifluoroacetyl, —C(O)Obenzyl (Cbz) or —C(O)O-tert.butyl (BOC), 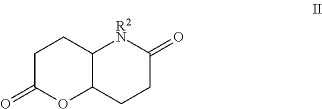
with a strong organic or inorganic base, preferably organic lithium or sodium amides as LDA or LiHMDS or NaHMDS, and then with either
aa) acetone followed by dehydration and isomerization step providing a compound of formula III, wherein R² is the same as defined for compound of formula II, 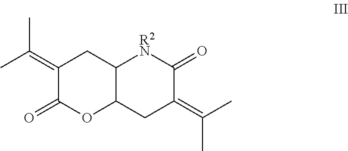
which is then subjected hydrogenation or reduction step, preferably heterogeneous hydrogenation over Pd or Pt catalyst, to reduce both double bonds or
bb) isopropyl halide, preferably bromide or iodide,
providing compound of formula IV, wherein R² is the same as defined for compound of formula II, 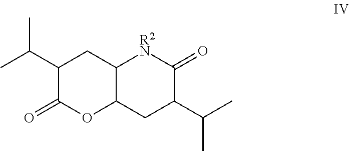
b) reaction of the compound of formula IV with a compound of formula V, 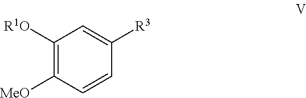
wherein R¹ is the same as defined in compound of formula I and R³ is a metal containing group such as —Li, —Na, -Mghalide, -Znhalide, cuprate or -Cuhalide or boronic acid, as e.g. —B(OH)₂ or -Cehalide providing, after eventual protection of C(4)-hydroxyl group, a compound of formula VI, wherein R¹, R² and R⁴ are the same as defined for compound of formula I; 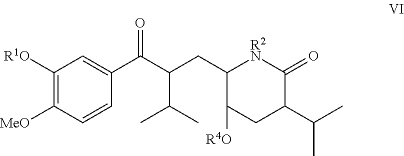
c) hydrogenation or/and reduction of C(8)-oxo group in the compound of formula VI, simultaneously or in two separate steps, to a compound of formula VII, wherein R¹, R² and R⁴ are the same as defined for compound of formula I; 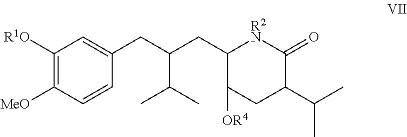
d) hydrolysis of the lactam of formula VII then eventual protection of C(5)-amino-group and either
aa) after protection of C(4)-hydroxyl group and activation of the free carboxylic acid function with a peptide coupling reagent, or
bb) formation of the 5-membered lactone ring with C(4)-hydroxyl group, followed by reaction with R⁵—H, wherein R⁵ is the same as defined for compound of formula I, preferably with NH₂CH₂C(CH₃)₂CONH₂.

12. A process according to the claim 11, wherein the compound of formula I has the configuration as given 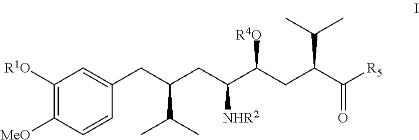
and the compounds of formulas II, III, IV, VI and VII have the configuration as defined in claims 2, 4, 6, 8 and 10, respectively.

13. A process for the preparation of a compound of formula IV as defined in claim 5 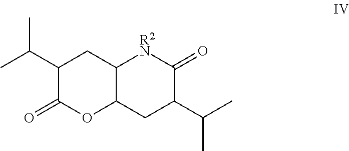
comprising reaction of the compound of formula II as defined in claim 1 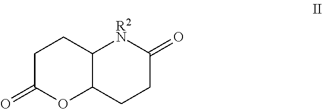
with a strong organic or inorganic base, preferably organic lithium or sodium amides as LDA or LiHMDS or NaHMDS, and then with either
a) acetone followed by dehydration and isomerization step providing a compound of formula III as defined in claim 3 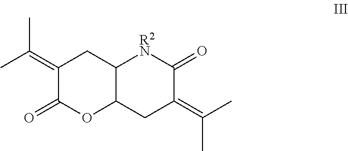
in which double bonds are subsequently hydrogenated or reduced to single bonds; or
b) isopropyl halide, preferably bromide or iodide.

14. A process according to claim 13, wherein the compound of formula IV is as defined in claim 6 and the compound of formula II is as defined in claim 2.

15. A process according to claim 14, wherein R² is hydrogen, benzyl, dimethoxybenzyl, trialkylsilyl, methoxy, benzyloxycarbonyl (Cbz), tert.-butyloxycarbonyl (BOC), formyl, acetyl and trifluoracetyl.

16. A process for the preparation of compound of formula VI, as defined in claim 7 comprising reaction of the compound of formula IV as defined in claim 5 with compound of formula V as defined in claim 11.

17. A process according to claim 16, wherein the compounds of formulas IV and VI are as defined in claims 6 and 8, respectively.

18. A process for the preparation of compound of formula VII, as defined in claim 9 or 10, comprising hydrogenation or/and reduction of C(8)-oxo group in compound of formula VI, as defined in claim 7 or 8 respectively, either simultaneously or in two separate reduction steps.

19. A process according to any one of claims 11-18, wherein in compounds of formulas I, II, III, IV, V, VI and VII
R¹ is CH₃OCH₂CH₂CH₂—,
R² is hydrogen, benzyl, methoxy, benzyloxy,
R³ is lithium or magnesium-chloride or bromide,
R⁴ is hydrogen,
R⁵ is hydroxy, lower alkoxy, benzyl or —NHCH₂C(CH₃)₂CONH₂.
